(19) United States
(12) Patent Application Publication
(10) Pub. No.: US 2024/0408079 A1
(43) Pub. Date: Dec. 12, 2024

(54) COMPOSITIONS AND METHODS FOR THE TREATMENT OF CANCER

(71) Applicant: THE TRANSLATIONAL GENOMICS RESEARCH INSTITUTE, Phoenix, AZ (US)

(72) Inventors: Sunil Sharma, Phoenix, AZ (US); Raffaella Soldi, Phoenix, AZ (US); Samuel Sampson, Cincinnati, OH (US)

(21) Appl. No.: 18/699,189

(22) PCT Filed: Oct. 20, 2022
(86) PCT No.: PCT/US2022/047319

§ 371 Date: Apr. 5, 2024

Related U.S. Application Data
(60) Provisional application No. 63/270,005, filed on Oct. 20, 2021.

(51) Int. Cl. A61K 31/495; A61K 31/496; A61K 31/506; A61K 31/519; A61P 35/00

(52) U.S. Cl. CPC A61K 31/495; A61K 31/496; A61K 31/506; A61K 31/519; A61P 35/00

(57) ABSTRACT

The present disclosure includes compounds and methods for treating a subject having a cancer, in particular. Ewing's Sarcoma. A method of treating Ewing's Sarcoma in a subject may comprise the steps of administering to the subject a therapeutically effective amount of a first pharmaceutical composition comprising SP-2577 and administering to the subject a therapeutically effective amount of a second pharmaceutical composition selected from the group consisting of Ruxolitinib, Dasatinib, Nilotinib, Imatinib, Bosutinib, and a drug targeting ABL2 pathway. The drug targeting ABL2 pathway may be an ABL2 knockdown drug, i.e., an ABL2 inhibitor.

### CROSS REFERENCE TO RELATED APPLICATIONS

This application claims the benefit of U.S. Provisional Application No. 63/270,005 entitled “Compositions and Methods for the Treatment of Cancer” which was filed Oct. 20, 2021, the entire disclosure of which is hereby incorporated herein by this reference.

### FIELD

The present invention is related to compositions and methods for treating cancer, and more particularly, is related to compositions and methods for treating Ewing's Sarcoma.

### BACKGROUND

Ewing's Sarcoma (ES) is a rare and aggressive pediatric bone tumor that is driven by a chromosomal translocation and fusion between EWS and FLI1 genes resulting in a chimeric protein. Nearly 85% of ES cases express the EWS/FLI protein which functions as a transcription factor and drives oncogenesis. EWS/FLI interacts with lysine-specific demethylase 1 (LSD1) that is highly expressed in pediatric sarcomas, to repress critical tumor suppressors.

Current therapeutic approaches rely on surgery and chemotherapies and/or radiotherapy. Overall, the five-year survival rate for localized ES is 62%, however, patients that relapse have a five-year survival rate of 15-30% (Ewing Tumor-Survival Rates). A potent reversible LSD1 inhibitor, SP-2577 (Seclidemstat), was recently developed that shows the ability to impair tissue culture cell viability in multiple ES cell lines. Although currently in clinical trials for ES treatment, preclinical studies have shown little antitumoral activity.

### SUMMARY

A need exists for an effective therapeutic strategy for treatment of Ewing's Sarcoma (ES). A more favorable therapeutic response to SP-2577 may be obtained by combining drugs that target multiple pathways and/or inhibit resistance mechanisms. Arrayed CRISPR screening allows identification of survival genes that can be candidates for molecularly targeted drugs. Synthego's CRISPR druggable library screen identified 103 genes that when down regulated resulted in increased SP-2577 cytotoxicity in the ES. Among those, ABL2 knockdown was shown to have a significant impact on the survival of ES treated with SP-2577 in vitro and in vivo.

Combination treatment of SP-2577 with drugs targeting ABL2 pathway showed high synergy at low dose ratio in vitro. This study highlights the potential for a combination treatment of SP-2577 in ES patients. Further, these observations support the capability of arrayed CRISPR screening to identify new therapeutic strategies in ES.

As such, there is a demonstrated need to further investigate existing pharmaceuticals for potential cytotoxicity in the ES. The present disclosure has identified compositions and methods for treating a subject having cancer, and in particular ES. The treatment or therapeutic agent may include a combination drug therapy comprising LSD1 inhibitor, SP-2577, and at least one of Ruxolitinib, Dasatinib, Nilotinib, Imatinib, Bosutinib, and a drug targeting ABL2 pathway.

In some aspects, the disclosure concerns methods of treating a subject having Ewing's Sarcoma, the method comprising the step of administering to the subject a therapeutically effective amount of a first pharmaceutical composition comprising SP-2577 and a second pharmaceutical composition comprising an ABL inhibitor. In some embodiments, the ABL inhibitor is selected from the group consisting of Ruxolitinib, Dasatinib, Nilotinib, Imatinib, and Bosutinib. In certain embodiments, the subject is human.

In some embodiments, the pharmaceutical composition comprises one or more pharmaceutically acceptable excipients. The first pharmaceutical composition and the second pharmaceutical composition may be administered concurrently. Alternately, the first pharmaceutical composition and the second pharmaceutical composition are administered sequentially.

Administration may be by any appropriate means. In some embodiments, one or both of the first pharmaceutical composition and the second pharmaceutical composition are administered as a gas or aerosol. In other embodiments, one or both of the first pharmaceutical composition and the second pharmaceutical composition are administered as solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, or elixirs.

In another aspect, the disclosure concerns methods of treating cancer in a subject, comprising the steps of: administering to the subject a therapeutically effective amount of SP-2577; and administering to the subject a therapeutically effective amount of a first pharmaceutical composition selected from the group consisting of Ruxolitinib, Dasatinib, Nilotinib, Imatinib, Bosutinib, and a drug targeting ABL2 pathway. In some embodiments, the subject is human.

In certain embodiments, the first pharmaceutical composition comprises one or more pharmaceutically acceptable excipients. The first pharmaceutical composition and the second pharmaceutical composition may be administered concurrently or sequentially.

In some embodiments, one or both of the first pharmaceutical composition and the second pharmaceutical composition are administered as a gas or aerosol. In other embodiments, one or both of the first pharmaceutical composition and the second pharmaceutical composition are administered as solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, or elixirs.

In yet other embodiments, the disclosure concerns pharmaceutical compositions for the treatment of Ewing's Sarcoma, the pharmaceutical composition comprising: a first active ingredient, wherein the first active ingredient is SP-2577; and a second active ingredient, wherein the second active ingredient is selected from the group consisting of Ruxolitinib, Dasatinib, Nilotinib, Imatinib, Bosutinib, and a drug targeting ABL2 pathway. Some embodiments further comprise at least one pharmaceutically acceptable carrier. Certain drugs targeting ABL2 pathway are ABL inhibitors.

The pharmaceutical composition may be of any suitable form. Some pharmaceutical compositions are formulated as a liquid, emulsion, or suspension. Other pharmaceutical compositions are formulated as a tablet, pill, pellet, capsule, capsule including liquid, or powder.

In further aspects, the disclosure concerns kits comprising a therapeutically effective amount of a first pharmaceutical composition comprising SP-2577 and a second pharmaceutical composition comprising an ABL inhibitor. In some embodiments, ABL inhibitors may be selected from the group consisting of Ruxolitinib, Dasatinib, Nilotinib, Imatinib, and Bosutinib.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIGS. 1-3 illustrate CRISPR Synergy: Screening and Analysis.

FIGS. 4A-4B illustrate CRISPR Synergy: in vitro and in vivo validation.

FIGS. 5A-5B illustrate CRISPR Synergy: in vitro and in vivo validation.

FIG. 6 illustrates qPCR validation of ABL2 KD.

FIGS. 7A-7B illustrate Westerns Focusing on Apoptosis markers and shows there is a ˜25% increase in Cleaved Caspase 3 seen in the combination treatment of siABL2 with 1 uM SP-2577.

FIG. 8 illustrates Westerns Examining MOA of ABL2i+SP-2577.

FIGS. 9A-9B illustrate CRISPR Synergy: in vitro and in vivo validation.

FIGS. 10A-10D illustrate CRISPR Synergy: in vitro and in vivo validation.

FIGS. 11A-11C illustrate the IC 50 (half maximal inhibitory concentration).

FIGS. 12A-12B illustrate CRISPR Synergy: in vitro and in vivo validation 2D.

FIGS. 13A-13B illustrate CRISPR Synergy: in vitro and in vivo validation 3D.

FIGS. 14A-14D illustrate Determination of FoxR2 KO in polyclonal population.

FIGS. 15A-15C illustrate In-cell Western Determines Positive KO Colonies.

FIGS. 16A-16B illustrate FoxR2 Western blot.

FIGS. 17A-17B illustrate FOXR2 shRNA silencing in breast cancer models.

FIGS. 18A-18C illustrate CRISPR+HDR plasmids co-transfection MCF-7.

FIGS. 19A-19C illustrate CRISPR+HDR plasmids co-transfection MB-468.

FIGS. 20A-20B illustrates the method of developing shRNA KO of TWIST and TRRAP COV434 inducible lines.

FIG. 21 illustrates TWIST and TRRAP screening.

FIGS. 22A-22B illustrate validation and growth curves after CRISPR transfection.

FIGS. 23A-23C illustrates examples of TWIST1 inhibitors.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

Inhibition encompasses any action that hinders, from any detectable level up to and including complete inactivation, the progression of a biological process. Such biological processes include expression of a gene or activities of a gene product, progression of a disease, normal and abnormal metabolic activities, interactions between entities within an organism, or interactions between one organism and another. Further nonlimiting examples of biological processes include development, death, maturation, infection, pain, apoptosis, or homeostasis. Inhibition includes actions that silence or repress the expression of a gene. Inhibition also includes actions that hinder the activity of the RNA product, protein product, or postranslationally modified protein product of a gene. Inhibition may be effectuated through a single agent that inactivates a single gene or gene product, by a single agent that inactivates a combination of more than one gene or gene product, a combination of agents that inactivates a single gene or gene product or a combination of agents that inactivates a combination of more than one gene or gene product.

Inhibition may be effectuated directly by an agent that directly causes the inhibition of a biological process or by agents that trigger one or more different biological processes to effectuate the inhibition of the first biological process. Agents that cause inhibition may also be called inhibitors. Examples of inhibitors include compositions such as compounds that trigger RNAi silencing such as microRNA or siRNA, antisense oligonucleotides, small molecular compounds, proteins such as soluble receptors or antibodies or any fragment thereof, including a Fab, F(ab)2, Fv, scFv, Fc, phage display antibody, peptibody or any other composition of matter that may inactivate or hinder a biological process.

In some embodiments, the one or more disclosed compounds can be synthesized using conventional techniques that may be well known to those skilled in the art. In other aspects, the one or more disclosed compounds can be purchased from one or more suppliers.

The concept of a pharmaceutical composition includes one or more of the disclosed compounds or a pharmaceutically acceptable salt thereof with or without any other additive/pharmaceutically acceptable excipient. The physical form of the invention may affect the route of administration and one skilled in the art would know to choose a route of administration that takes into consideration both the physical form of the compound and the disorder to be treated. Pharmaceutical compositions that include the disclosed compounds may be prepared using methodology well known in the pharmaceutical art. A pharmaceutical composition that includes one or more of the disclosed compounds may include a second effective compound of a distinct chemical formula from the disclosed compounds. This second effective compound may have the same or a similar molecular target as the disclosed compounds or it may act upstream or downstream of the molecular target of the disclosed compounds with regard to one or more biochemical pathways.

Pharmaceutical compositions, including the one or more disclosed compounds, may include materials capable of modifying the physical form of a dosage unit (e.g., pharmaceutically acceptable excipients). In one non-limiting example, the composition includes a material that forms a coating that contains the one or more disclosed compounds. Materials that may be used in a coating include, for example, sugar, shellac, gelatin, or any other inert coating agent.

Pharmaceutical compositions including the one or more disclosed compounds may be prepared as a gas or aerosol. Aerosols encompass a variety of systems including colloids and pressurized packages. Delivery of a composition in this form may include propulsion of a pharmaceutical composition including the one or more disclosed compounds through use of liquefied gas or other compressed gas or by a suitable pump system. Aerosols may be delivered in single phase, bi-phasic, or multi-phasic systems.

In some aspects of the invention, the pharmaceutical composition including the one or more disclosed compounds is in the form of a solvate. Such solvates are produced by the dissolution of the one or more disclosed compounds in a pharmaceutically acceptable solvent. Pharmaceutically acceptable solvents include any mixtures of one or more solvents. Such solvents may include pyridine, chloroform, propan-1-ol, ethyl oleate, ethyl lactate, ethylene oxide, water, ethanol, and any other solvent that delivers a sufficient quantity of the one or more disclosed compounds to treat the indicated condition.

Pharmaceutical compositions that include the one or more disclosed compounds may also include at least one pharmaceutically acceptable carrier/excipient. As used herein, “carrier(s)” can be used interchangeably with “excipient(s)” Carriers include any substance that may be administered with the one or more disclosed compounds with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils.) Such carriers include particulates such as a tablet or powder, liquids such as oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic acid, sodium alginate. Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carrier that may be formulated into a capsule. Still further examples of carriers include sterile diluents such as water for injection, saline solution, physiological saline. Ringer's solution, isotonic sodium chloride, fixed oils such as synthetic mono or diglycerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

The pharmaceutical composition, including the one or more disclosed compounds, may take any of a number of formulations depending on the physicochemical form of the composition and the type of administration. Such forms include solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs, or any other formulation now known or yet to be disclosed. Additional examples of suitable pharmaceutical carriers and formulations are well known in the art.

Methods of administration include, but are not limited to, oral administration and parenteral administration. Parenteral administration includes, but is not limited to intradermal, intramuscular, intraperitoneal, intravenous, subcutaneous, intrathecal, intranasal, epidural, sublingual, intramsal, intracerebral, intraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration of the disclosed compound to the area in need of treatment (e.g., areas of the respiratory tract, including the nasal cavity, the trachea, the lungs, the bronchi, etc.). Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include the use of an inhaler or nebulizer, formulation with an aerosolizing agent, or via perfusion in a fluorocarbon or synthetic pulmonary surfactant. The disclosed compound may be delivered in the context of a vesicle such as a liposome or any other natural or synthetic vesicle. Additional examples of suitable modes of administration are well known in the art.

A pharmaceutical composition formulated to be administered by injection may be prepared by dissolving the one or more disclosed compounds with water so as to form a solution. In addition, a surfactant may be added to facilitate the formation of a homogeneous solution or suspension. Surfactants include any complex capable of non-covalent interaction with the disclosed compound so as to facilitate dissolution or homogeneous suspension of the compound.

Pharmaceutical compositions including the one or more disclosed compounds may be prepared in a form that facilitates topical or transdermal administration. Such preparations may be in the form of a solution, emulsion, ointment, gel base, transdermal patch or iontophoresis device. Examples of bases used in such compositions include petrolatum, lanolin, polyethylene glycols, beeswax, mineral oil, diluents such as water and alcohol, and emulsifiers and stabilizers, thickening agents, or any other suitable base now known or yet to be disclosed.

Determination of an effective amount of the one or more disclosed compounds is within the capability of those skilled in the art, especially in light of the detailed disclosure provided herein. The effective amount of a pharmaceutical composition used to affect a particular purpose as well as its toxicity, excretion, and overall tolerance may be determined in vitro, or in vivo, by pharmaceutical and toxicological procedures either known now by those skilled in the art or by any similar method yet to be disclosed. One example is the in vitro determination of the IC₅₀ (half maximal inhibitory concentration) of the pharmaceutical composition in cell lines or target molecules. Another example is the in vivo determination of the LD₅₀ (lethal dose causing death in 50% of the tested animals) of the pharmaceutical composition. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for administration also includes the determination of an effective therapeutic amount and a pharmaceutically acceptable dose, including the formulation of an effective dose range for use in vivo, including in humans.

Treatment of a condition, such as treatment of a subject having cancer, is the practice of any method, process, or procedure with the intent of halting, inhibiting, slowing or reversing the progression of a disease, disorder, infection, or condition, substantially ameliorating clinical symptoms of a disease, disorder, infection, or condition substantially preventing the appearance of clinical symptoms of a disease, disorder or condition, up to and including returning the diseased entity to its condition prior to the development of the disease. Generally, the effectiveness of treatment is determined by comparing treated groups with non-treated groups.

The addition of a therapeutically effective amount of the one or more disclosed compounds encompasses any method of dosing of a compound. Dosing of the disclosed compound may include single or multiple administrations of any of a number of pharmaceutical compositions that include the one or more of the disclosed compounds as an active ingredient or active ingredients. Examples include a single administration of a slow release composition, a course of treatment involving several treatments on a regular or irregular basis, multiple administrations for a period of time until a diminution of the disease state is achieved, preventative treatments applied prior to the instigation of symptoms, or any other dosing regimen known in the art or yet to be disclosed that one skilled in the art would recognize as a potentially effective regimen. A dosing regimen including the regularity of and mode of administration will be dependent on any of a number of factors including but not limited to the subject being treated; the severity of the condition; the manner of administration, the stage of disease development, the presence of one or more other conditions such as pregnancy, infancy, or the presence of one or more additional diseases; or any other factor now known or yet to be disclosed that affects the choice of the mode of administration, the dose to be administered and the time period over which the dose is administered.

Regardless of the route of administration, the therapeutic agent can be typically administered at a daily dosage of 0.01 mg to 30 mg/kg of body weight of subject receiving the treatment (e.g., 1 mg/kg to 5 mg/kg). The pharmaceutical formulation can be administered in multiple doses per day, if desired, to achieve the therapeutic effect and/or the total desired daily dose.

Pharmaceutical compositions that include the one or more disclosed compounds may be administered prior to, concurrently with, or after administration of additional or second (or third) pharmaceutical compositions that may or may not include the one or more disclosed compounds. Concurrent administration refers to pharmaceutical compositions that may be administered within about one minute of each other. If not administered concurrently, the additional or second pharmaceutical compositions may be administered a period of one or more minutes, hours, days, weeks, or months before or after the pharmaceutical composition that includes the one or more disclosed compounds. Alternatively, a combination of pharmaceutical compositions may be cyclically administered. Cycling therapy involves the administration of one or more pharmaceutical compositions for a period of time, followed by the administration of one or more different pharmaceutical compositions for a period of time and repeating this sequential administration. Cycling therapy may be used, for example, to reduce the development of resistance to one or more of the compositions, to avoid or reduce the side effects of one or more of the compositions, and/or to improve the efficacy of the treatment.

Pharmaceutical compositions including the one or more disclosed compounds may be used in methods of treating a subject having Ewing's Sarcoma. Such methods involve the administration of an effective amount of a pharmaceutical composition that includes the one or more disclosed compounds and/or a pharmaceutically acceptable salt thereof to a subject with cancer (e.g., a mammal, such as a human or animal).

As used herein, the term “subject” or “patient” refers to any organism that is capable of developing cancer. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human.

The following examples are given for illustrative and non-limiting purposes of the present invention.

### Examples

Ewing's Sarcoma (ES) is a type of primary bone tumor affecting children and adolescents and accounts for 1% of all childhood cancers. This rare and aggressive disease is characterized by the t-chromosomal translocation of EWS/ETS resulting in a pathognomonic chimeric fusion gene, EWSR1-FLI1, which encodes the EWS-FLI protein in roughly 85% of all diagnosed cases.

Developing targeted therapies for ES has proved to be challenging. On one side, targeting EWS/FLI remains unfulfilled due to its intrinsically disordered protein nature and lack of enzymatic activity. On the other side, large-scale genomic sequencing has shown that ES possesses one of the lowest mutation rates amongst all cancers (0.15 mutations/Mb), drastically reducing the identification of potential therapeutic targets. EWS-FLI drives ES tumorigenicity through mechanisms that increase genome-wide chromatin accessibility at GGAA microsatellite repeats (20827386) by interacting with epigenetics complexes and creating de novo enhancer elements that establish the ES transcriptional signature. In particular, EWS/FLI recruits lysine specific demethylase 1 (LSD1) to the nucleosome remodeling and histone deacetylase complex (NuRD) causing a repression of the tumor suppressor genes LOX and TGFBR2 (23178492). LSD1 is a well-characterized histone lysine demethylase belonging to the family of flavin-dependent amine oxidases, which has an important role in stem cell maintenance through transcriptional repression.

Examination of LSD1 expression in various malignancies shows an increased expression in sarcomas including ES, desmoplastic small round cell tumors (DSRCT) chondrosarcoma, osteosarcoma, rhabdomyosarcoma, and synovial. Given the role played in EWS/FLI-dependent gene transcription, LSD1 has been aggressively pursued as a potential therapeutic target.

A potent reversible LSD1 inhibitor, SP-2577 (Seclidemstat) was recently developed. In preclinical studies SP-2577, and its tool compound SP-2509, have shown to reverse the transcriptional profiles driven by EWS-FLI and significantly delay tumorigenesis in vitro (24963049; 29997151). Although the LSD1 inhibitor is currently in phase 1 clinical trials for multiple cancer types including ES (NCT03600649), preclinical studies have shown that SP-2577 has had little antitumoral activity in vivo. To date, the use of SP-2577 as a therapeutic strategy for ES in clinics has not been comprehensively assessed.

However, combining SP-2577 with drugs that target multiple pathways and/or inhibit resistance mechanisms may result in a more favorable therapeutic response in ES patients. Over the last decade genome engineering tools such as RNA interference (RNAi) and Clustered Regularly Interspaced Short Palindromic Repeats (CRISPR) have made massive strides in advancement. CRISPR libraries for loss of function have been widely used to identify new biological mechanisms, such as drug resistance and cell survival signals. CRISPR negative selection is capable of detecting dead or slow-growing cells efficiently, and it can identify survival-essential genes, which can be promising candidates for molecularly targeted drugs.

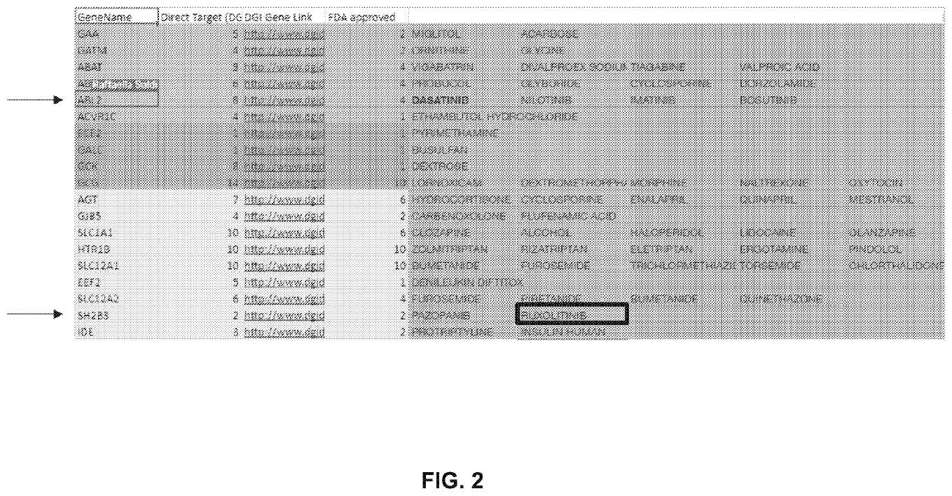

In this study, by applying the Synthego CRISPR genome-wide arrayed library to ES cell lines treated with SP-2577, the inventors identified 103 candidate synergistic genes (FIG. 2). Among the top ranking genes, ABL2 (Abelson Murine Leukemia Viral Oncogene Homolog 2) down regulation significantly impacted SP-2577 efficacy in ES in vitro and in vivo. ABL2, also referred to as ABL related gene (ARG), is an oncogene associated with chromosome translocations in human leukemias which results in constitutively activated forms of the ABL2 tyrosine kinase required for cellular transformation.

Recently, activation of ABL kinases ABL1 and ABL2 has been detected in solid tumors, including breast, colon, lung, prostate, clear cell renal carcinoma, and melanoma. Activation of ABL1 and ABL2 in solid tumors is not linked to chromosome translocation events, but rather is driven by enhanced ABL kinases expression and/or by oncogenic tyrosine kinases, chemokine receptors, oxidative stress, and/or inactivation of negative regulatory proteins.

The presently-disclosed show that down regulation of ABL2 expression affects the tumorigenicity of ES cells in vitro. Further, co-treatment of SP-2577 with FDA approved ABL2 inhibitors is synergistic in ES in vivo and ex-vivo models. Our study suggests a potential new clinical treatment regimen for patients with ES. Additionally, the Synthego CRISPR platform has been demonstrated as an efficient tool to identify novel combinations of targeted drugs opening new horizons for more effective treatment of tumors.

### Methods

### CRISPR Screening Identifies Candidate SP-2577 Synergistic Genes in Ewing's Sarcoma: ABL2.

To identify genes regulating SP-2577 toxicity, the inventors conducted an arrayed CRISPR screen on SKNMC ES cell line using a pre-designed standard library that includes approximately 8,000 genes known to be part of druggable pathway. Instead of a typical “pooled” screening approach, where guide RNAs (gRNAs) for multiple genes are delivered together, the inventors used an “arrayed” screening approach, which comprises pools of at least three high-quality synthetic gRNAs (sgRNAs) per gene targets, mostly targeting 5′ protein-coding exons, delivered as one-gene-per-well pools for efficient knockout.

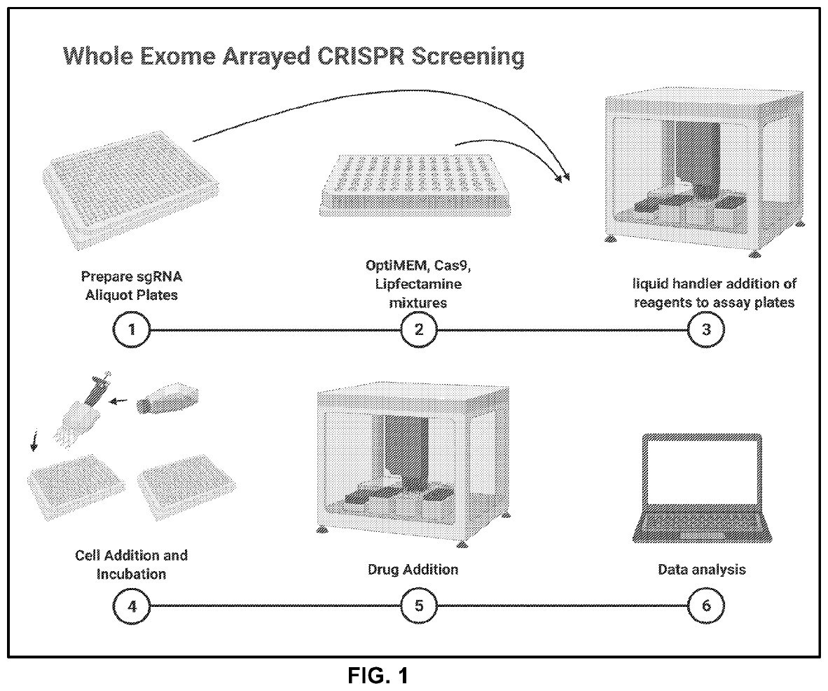

FIG. 1 shows the experimental workflow for our screening. Briefly, ES cells were transfected with the CRISPR Synthego arrayed library in two identical 384 well plates and allowed to incubate for 48 hours. Plates were then subjected to either SP-2577 treatment or untreated, and after 72 h incubation with SP-2577, cell viability levels were assessed by Cell Titer Glo assay.

To identify hit genes, the inventors used custom R code described in the materials and methods to compare growth between the experimental condition relative to an untreated control using Z-score.

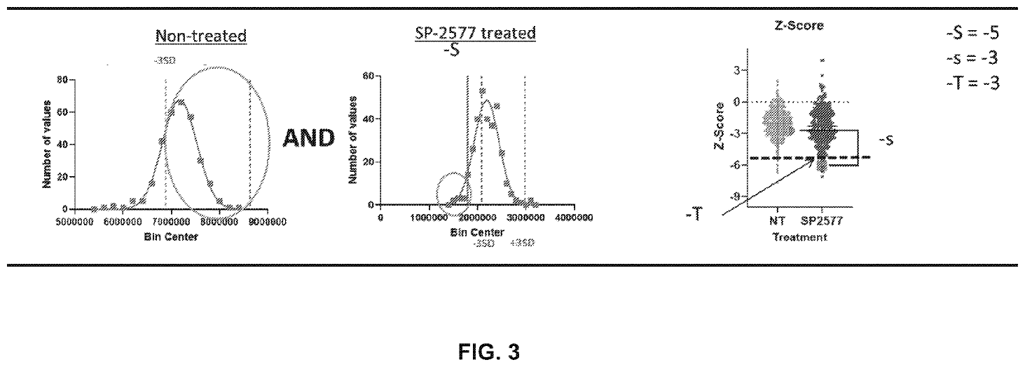

To ensure consistency across plates, SP-2577 inhibition calculation, control well quality control steps, and Z-factor calculation were used. A Z-factor cut-off ≥0.5 was selected to define the lower boundary to describe an excellent assay (FIG. 3).

To identify synergistic combinations of drug targets in ES cells, the inventors selected genes with lower Z-score that fit a number of criteria. First, the inventors chose genes with corresponding drugs in DrugBank, PharmGKB, Chembl, Drug Target Commons, TTD.

To enrich synergistic pairs, the inventors removed genes with lethal single-gene-deletion phenotypes as their phenotypes would not be further aggravated by additional gene deletions. For the 103 genes that met these criteria, the inventors hypothesized that drugs targeting these genes would be toxic to ES cells in combination with SP-2577.

To test the generality of the findings, the inventors validated hit genes from the primary screen performed on the ES cells that showed strength of the phenotype from the CRISPRi screen, Ingenuity pathway analysis (IPA) results on the interaction with LSD1 signaling, and potential as therapeutic targets. Among those, ABL2 showed a Z-score of −5.02 in the CRISPRi screen for ABL2 knockout, with a decrease of survival compared to the untreated SKNMC cell line. IPA analysis revealed a possible connection between LSD1 pathway and ABL2 through HOTAIR pathway. lncRNA HOTAIR is consistently expressed among several tumors and induces epigenetic and gene expression changes that cooperate in tumorigenesis.

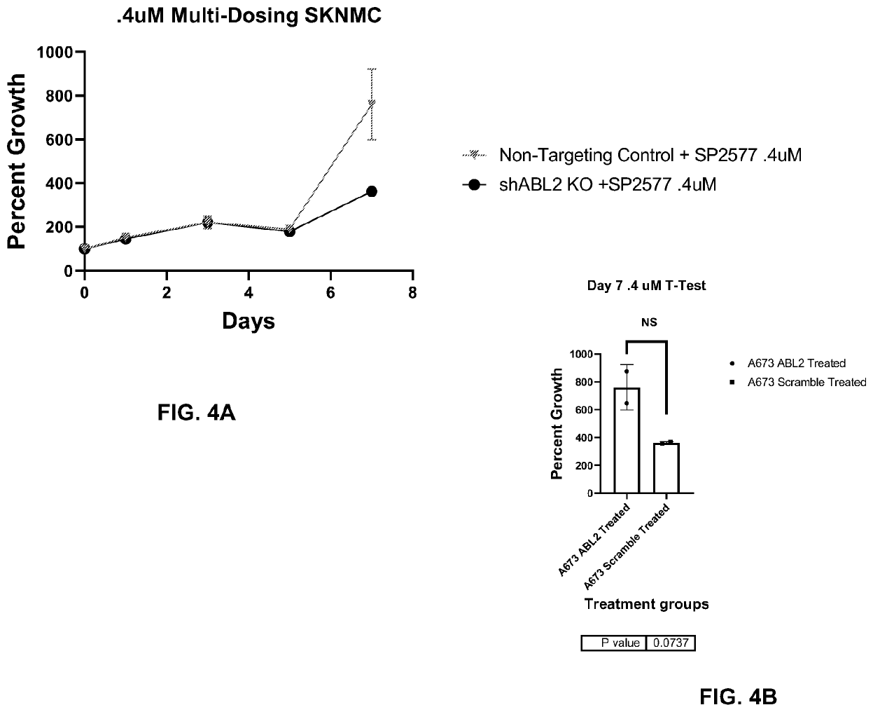

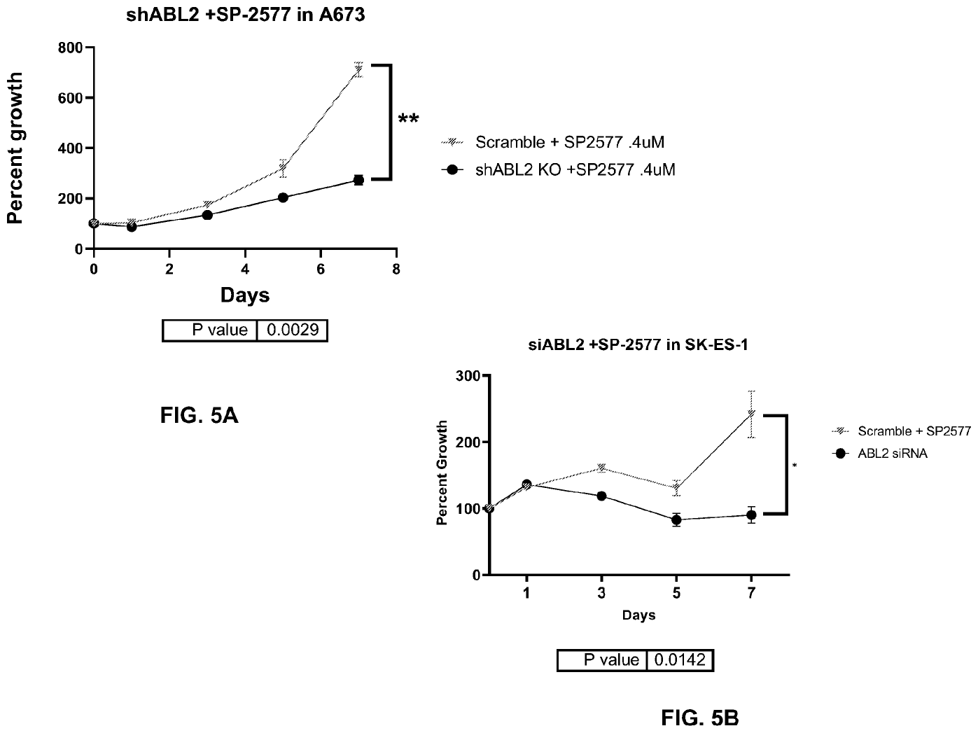

FIGS. 4A, 4B, 5A and 5B show percentage growth over time for multi-dosing SKNMC, shABL2 plus SP-2577 in A673 and for siALB2 plus SP2577 in SK-ES-1.

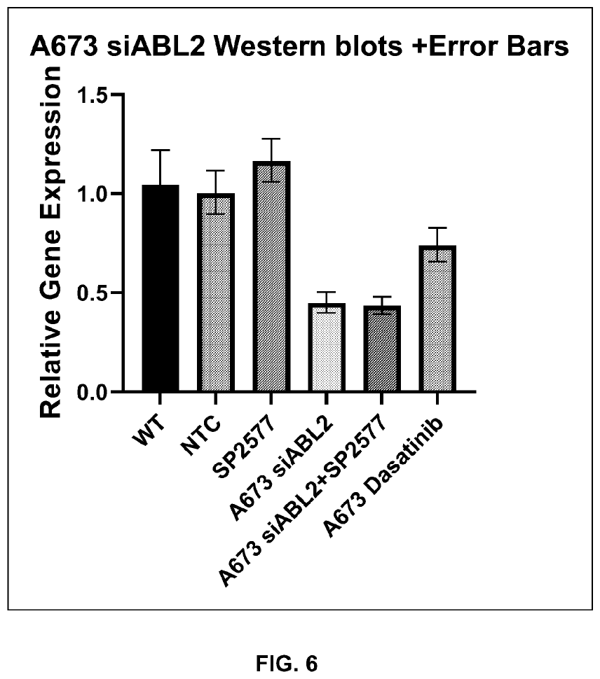

FIG. 6 presents results for A673 siABL2 Wester Blots for WT, NtC, SP2577, A673 siABL2, A673 siABL2+SP2577, and A673 Dasatinib. Error bars are shown for each bar.

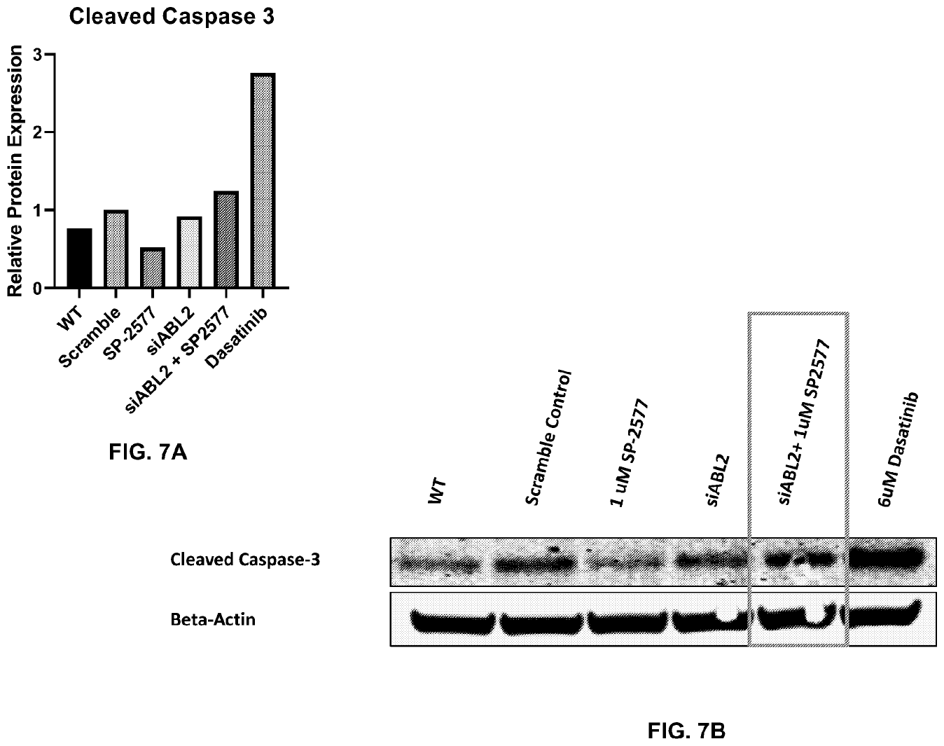

FIGS. 7A and 7B show relative protein expression for WT, Scramble, SP-2577, siABL2, siABL2+SP2577, and Dasatinib for cleaved Caspase 3 protein.

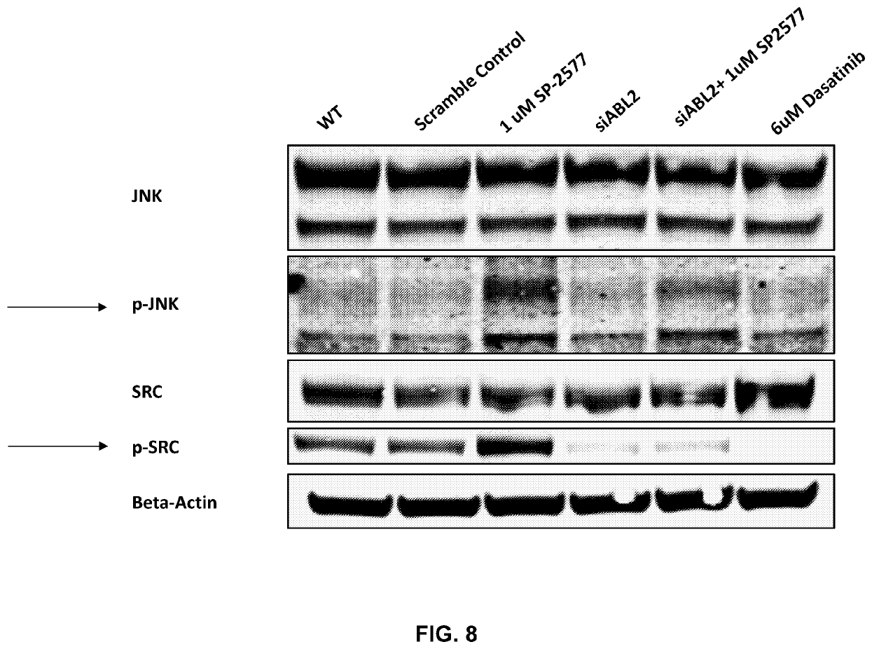

FIG. 8 illustrates Westerns Examining MOA of ABL2i+SP-2577.

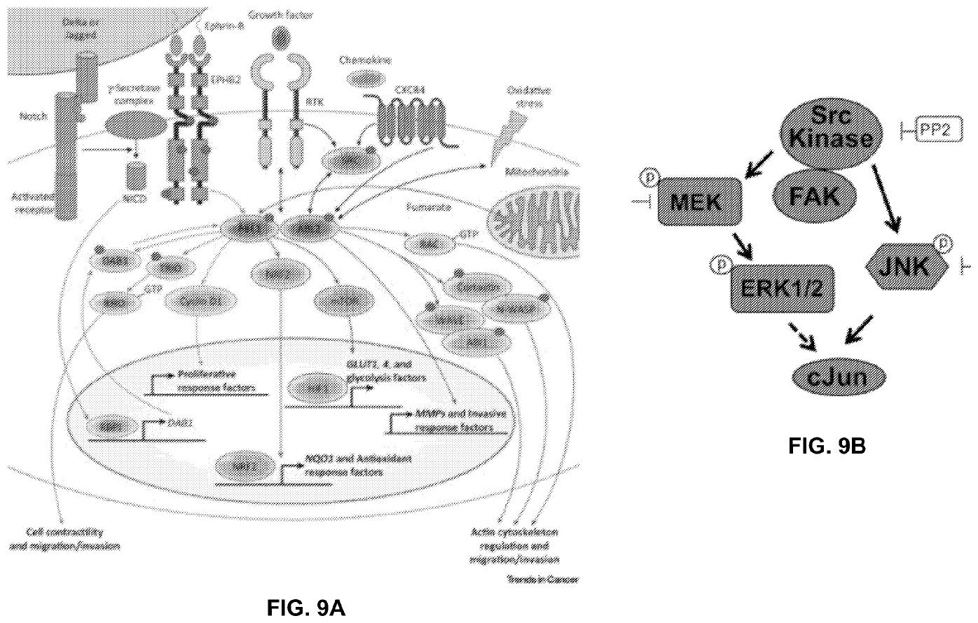

FIGS. 9A and 9B illustrate CRISPR Synergy: In vitro and In vivo validation.

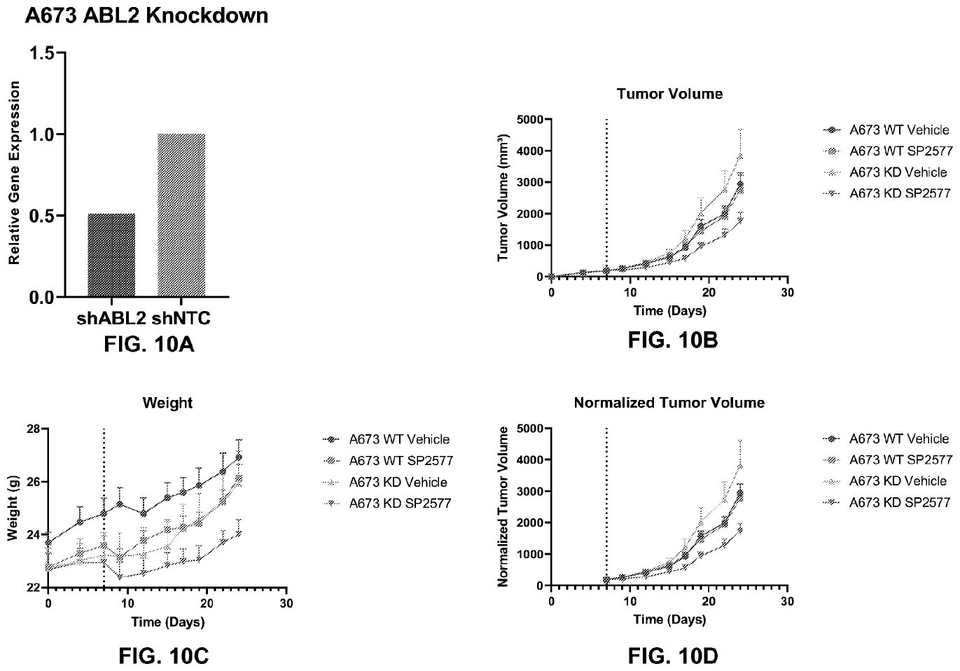

FIG. 10A presents relative gene expression for shABL2 and shNTC. FIGS. 10B-10D utilize the A673 cell line and show tumor volume, weight and normalized tumor volume for A673 WT Vehicle, A673 WE SP2577, A673 KD Vehicle, and A673 KD SP2577.

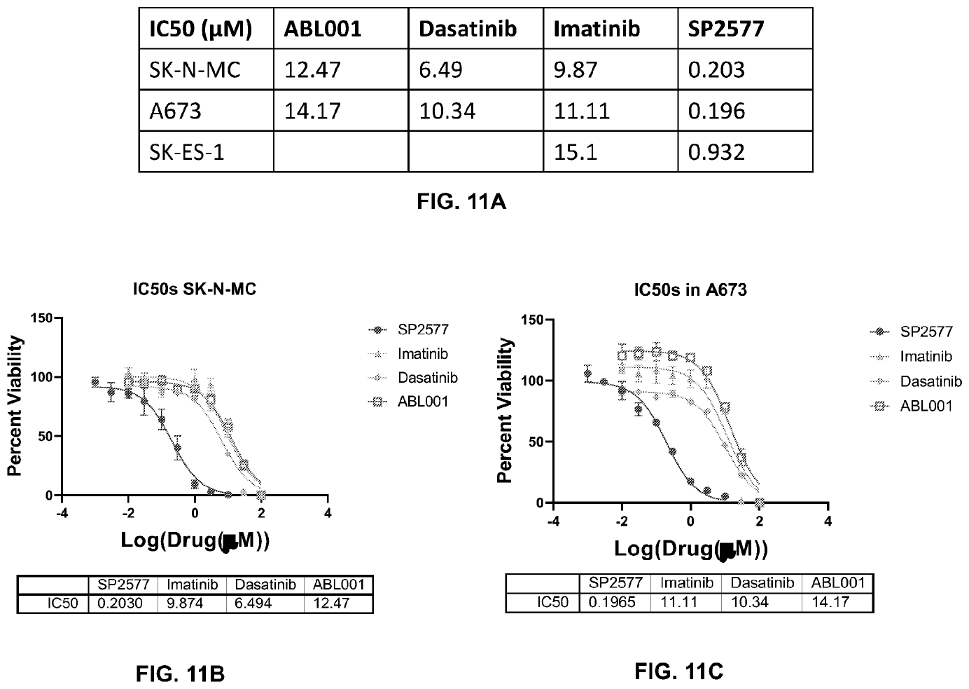

FIG. 11A presents IC50 (μM) for ABL001, Dasatinib, Imatinib and SP2577 for SK-N-MC A673 and SK-ES-1 cell lines. FIGS. 11B and 11C present graphs of percent viability of ABL001, Dasatinib, Imatinib and SP2577 for SK-N-MC and A673 cell lines.

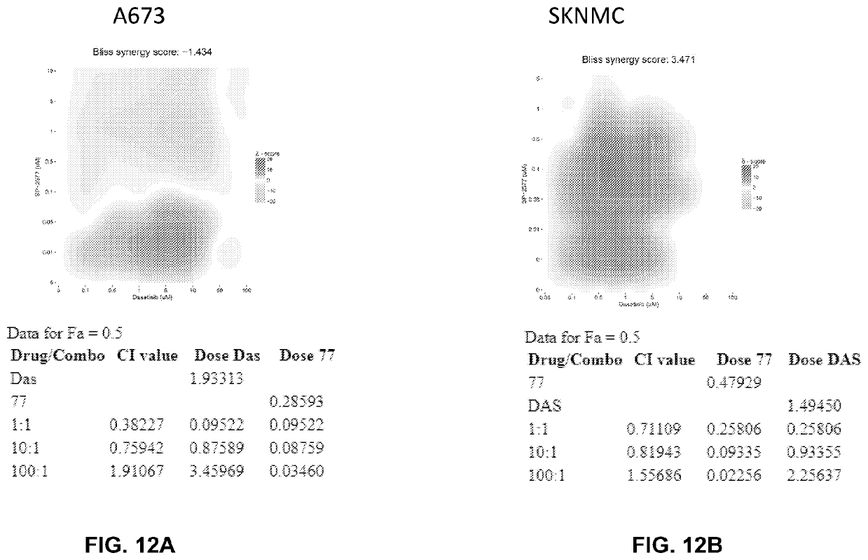

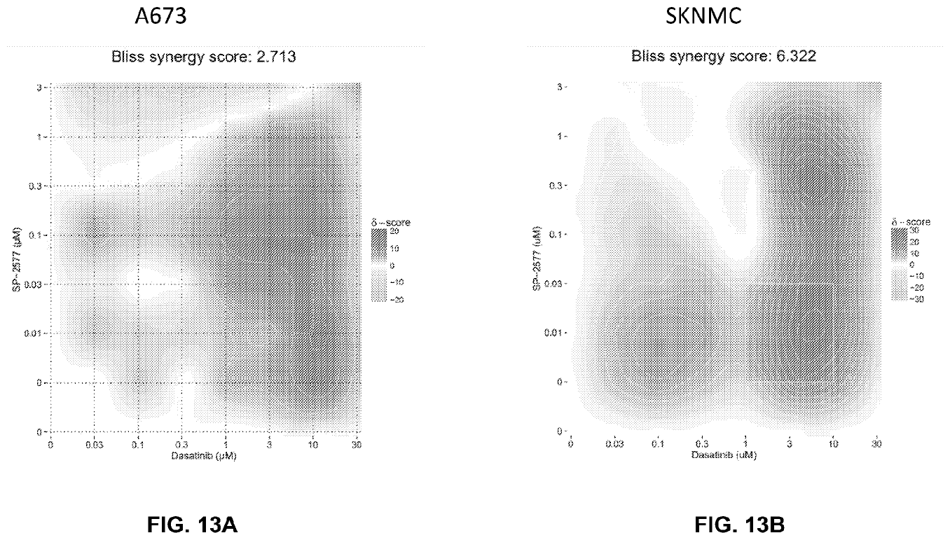

FIGS. 12A and 12B present Bliss synergy scores of −1.434 for SP-2577 and Dasatinib in A673 and 3.471 for SP-2577 and Dasatinib in SKNMC. In FIGS. 13A and 13B the results for the same combinations were 2.713 and 6.322 respectively. Such scores indicate that effect of the combination of drugs is additive.

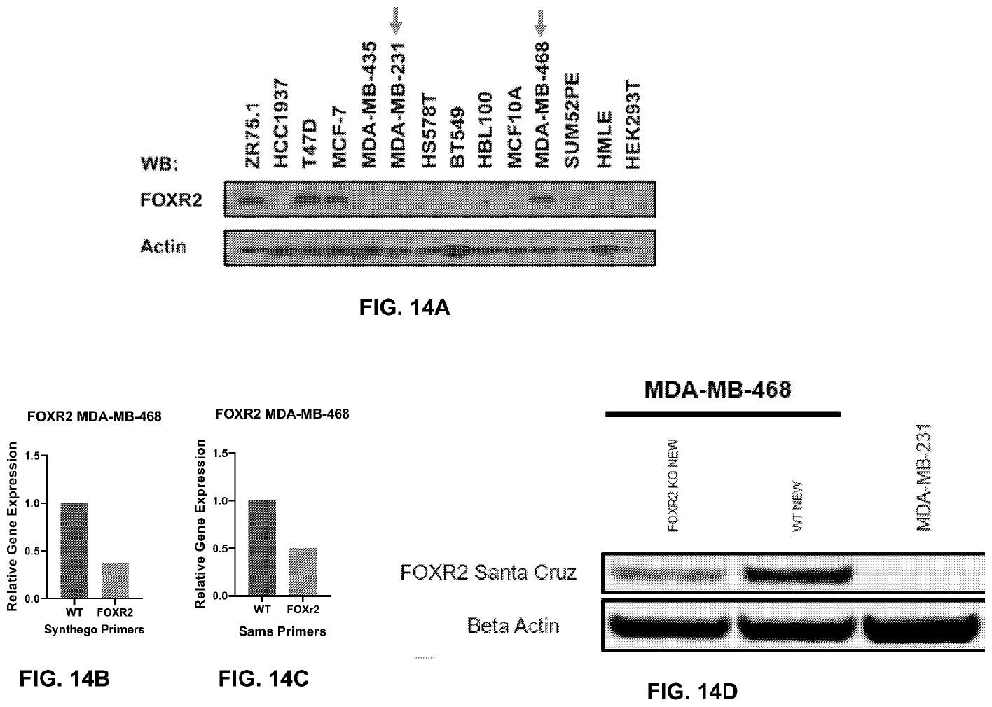

FIGS. 14A-14D illustrate determination of FoxR2 KO in polyclonal population for various epithelial cell lines (ZR75.1, HCC1937, T47D, MDA-MB-435, MDA-MB-231, HS578T, BT-549, HBL100, MCF10A, MDA-MB-468, SUM52PE, HMLE, and HEK293T). FOX R2 (Forkhead Box R2) is a gene silencer. Beta Actin is often used in Western Blot studies to normalize protein amounts and check for protein degradation.

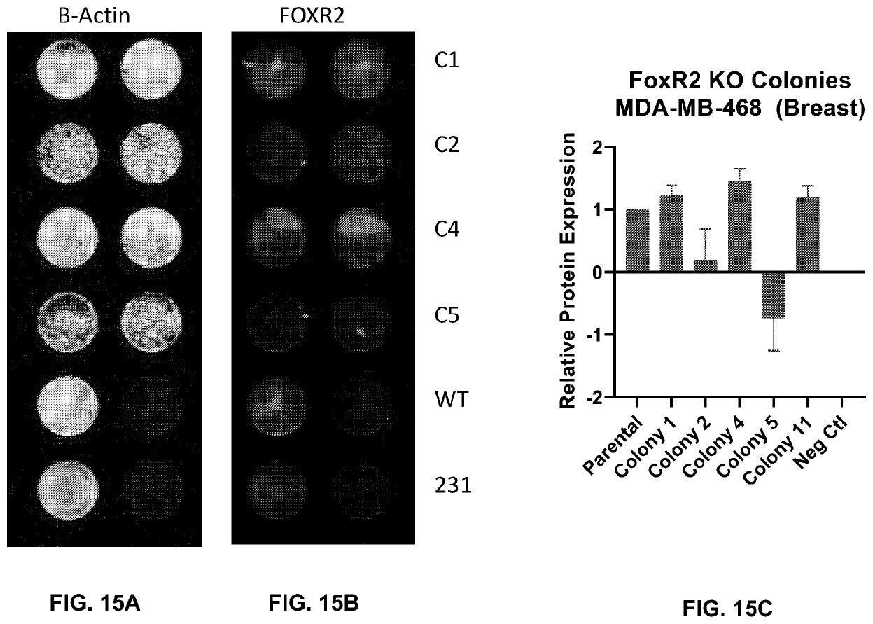

FIGS. 15A-15C illustrate in-cell positive KO colonies for various FoxR2 colonies of MDA-MB-468 (breast) and a negative control.

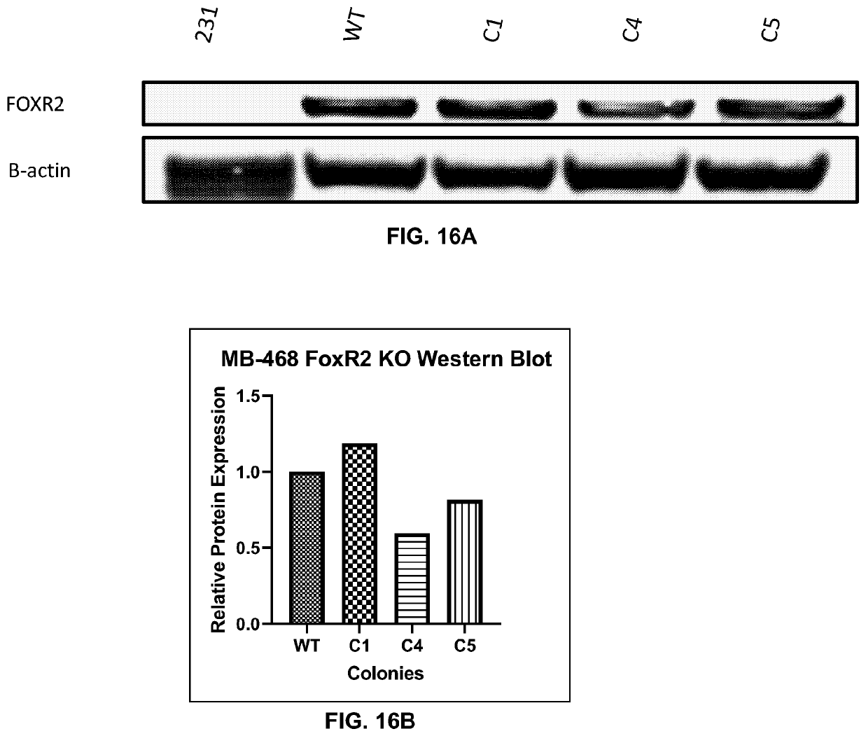

FIGS. 16A-16B illustrate MD-468 FoxR2 western blot. Relative protein expression of various colonies are shown.

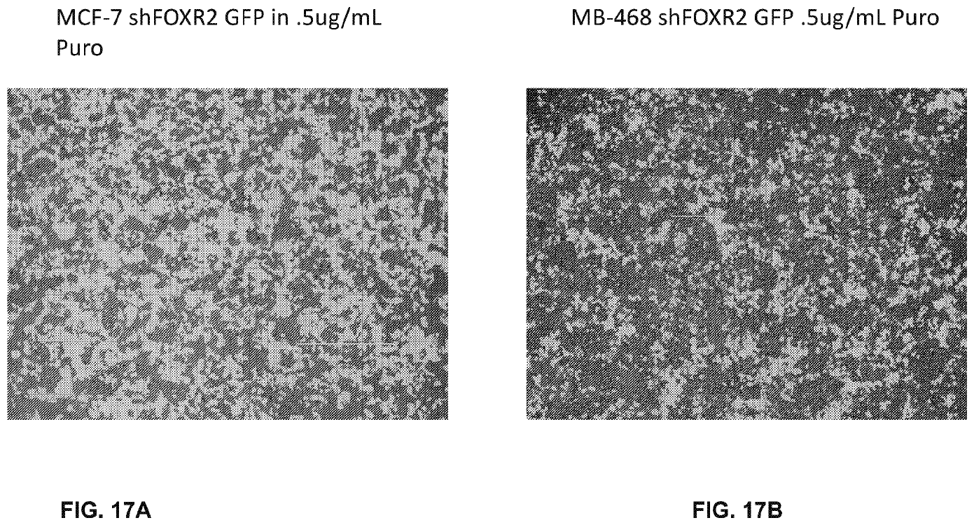

FIGS. 17A-17B illustrate FOXR2 shRNA silencing in breast cancer models. FIG. 17A shows results for MCF-7 shFOXR2 GFP in 0.5 ug/mL Puro. MCF-7 is a human breast cell line. GFP is green fluorescent protein that exhibits a bright green fluorescence when exposed to blue or IV light. FIG. 17B shows results for MB-468 shFOXR2 GFP 0.5 ug/mL Puro. MB-468 is a human breast cell line.

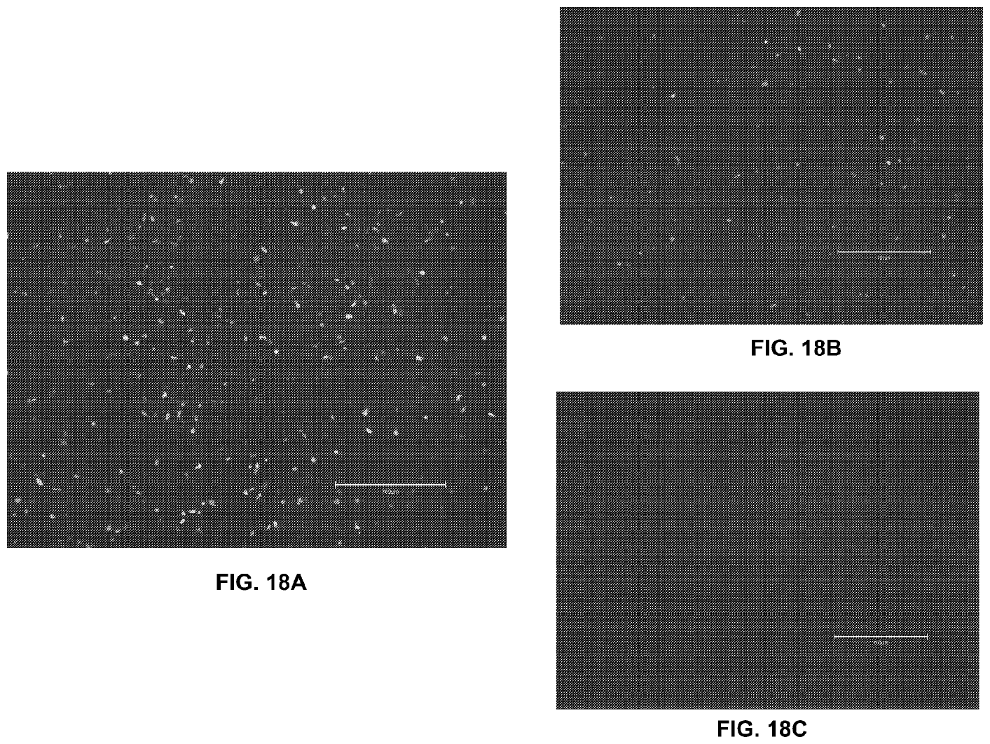

FIGS. 18A-18C illustrate CRISPR+HDR plasmids co-transfection MCF-7, a cell line of epithelial cells from breast cancer tissue form a patient with metastatic adenocarcinoma.

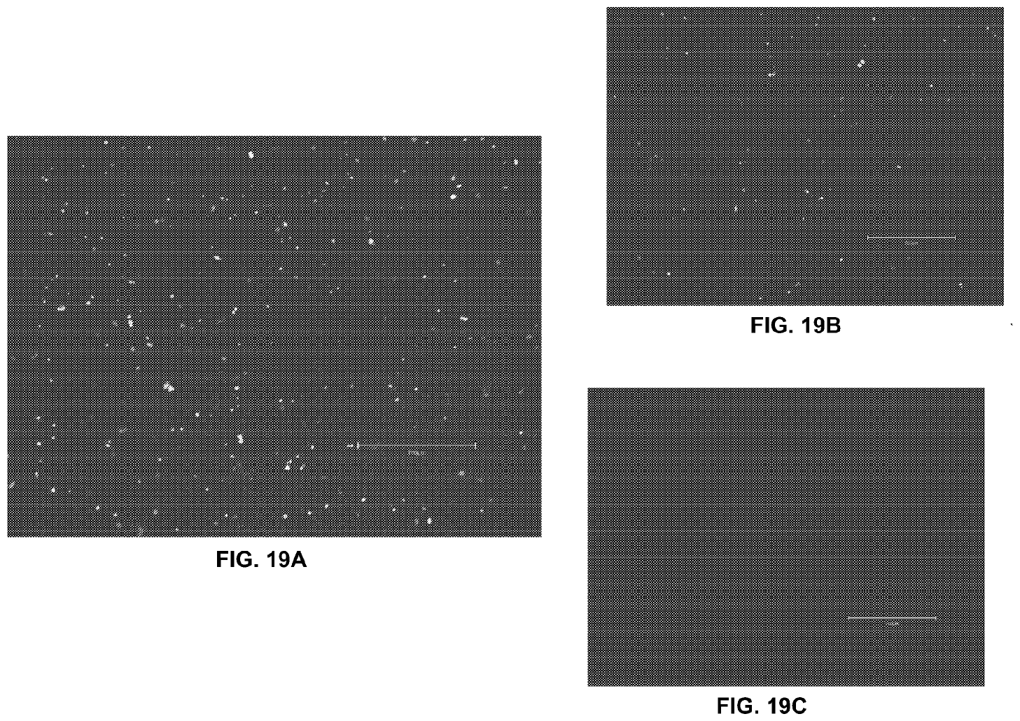

FIGS. 19A-19C illustrate CRISPR+HDR plasmids co-transfection MB-468.

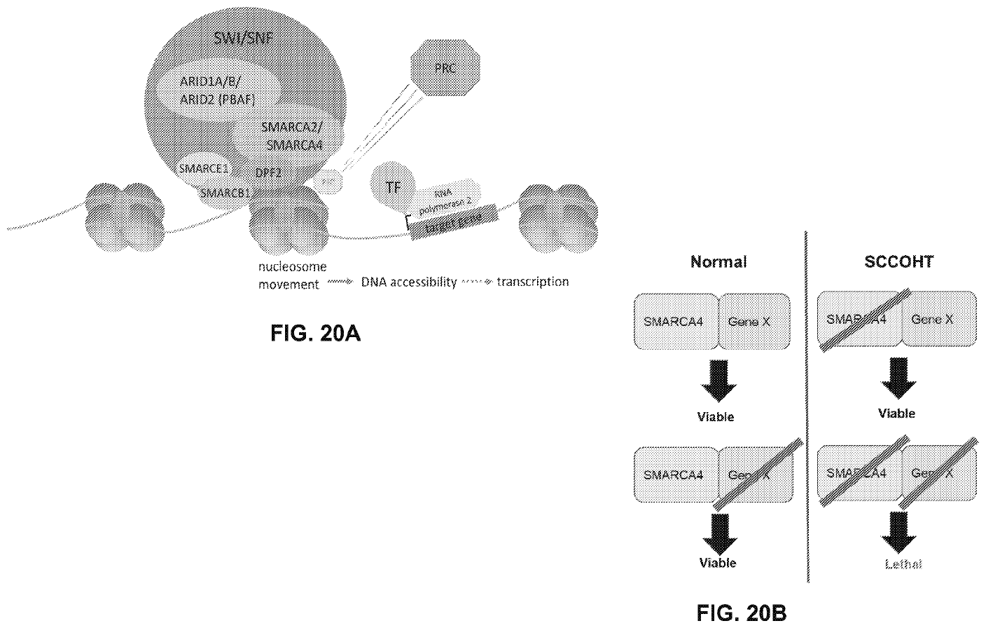

FIGS. 20A-20B illustrates the method of developing shRNA KO of TWIST and TRRAP COV434 inducible lines.

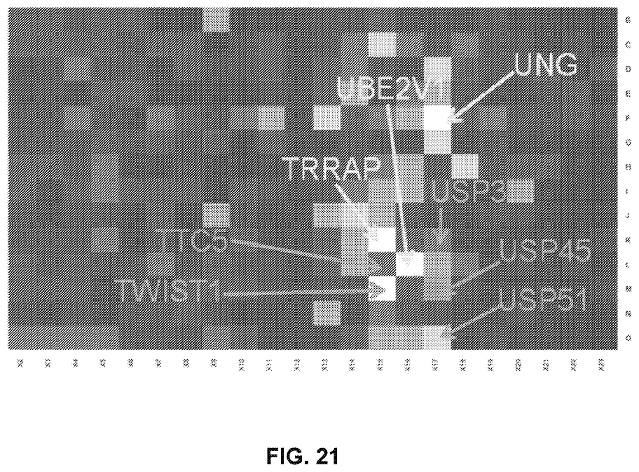

FIG. 21 illustrates TWIST and TRRAP screening.

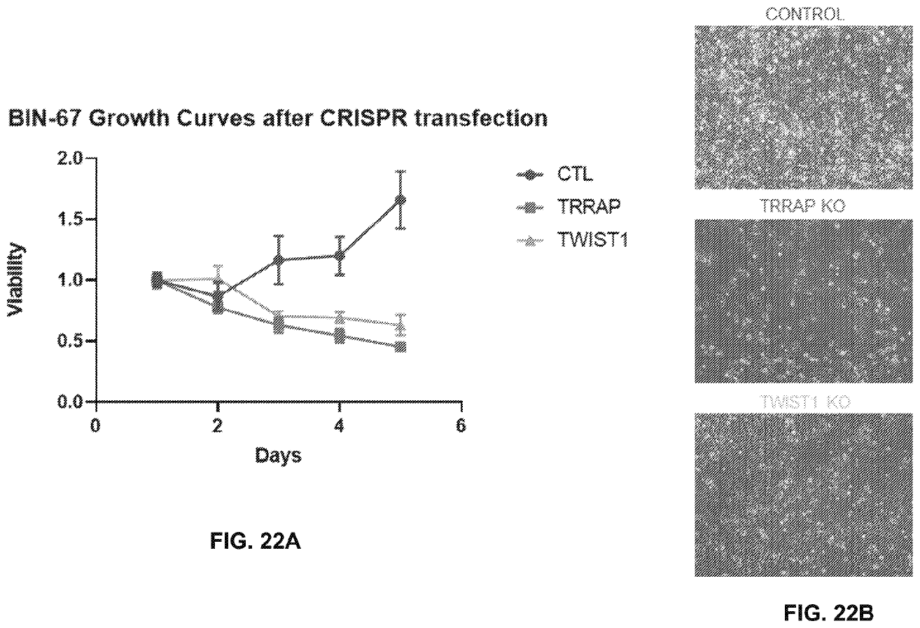

FIGS. 22A-22B illustrate validation and growth curves after CRISPR transfection. Control, TRRAP KO and TWIST1 KO are shown in FIG. 22B. TRRAP is Transformation/Transcription Domain Associated Protein. TWIST1 is Twist-related Protein 1.

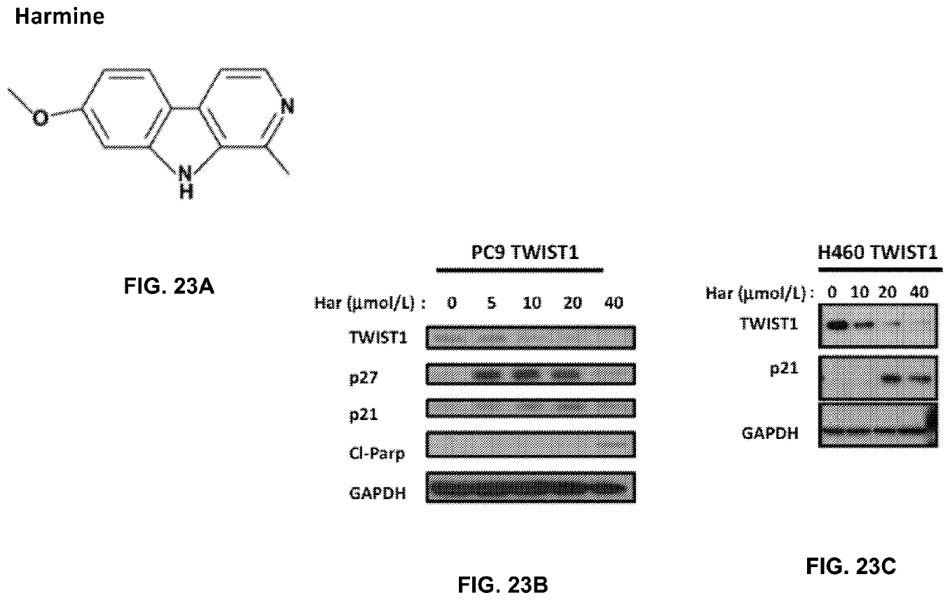

FIGS. 23A-23C illustrates examples of TWIST1 inhibitors. Level of Harmine (depicted in FIG. 23A) is shown in the FIG. 23B and FIG. 23C.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a subject having Ewing's Sarcoma, the method comprising the step of administering to the subject a therapeutically effective amount of a first pharmaceutical composition comprising SP-2577 and a second pharmaceutical composition comprising an ABL inhibitor.

2. The method of claim 1, wherein the ABL inhibitor is selected from the group consisting of Ruxolitinib, Dasatinib, Nilotinib, Imatinib, and Bosutinib.

3. The method of claim 1, wherein the subject is human.

4. The method of claim 1, wherein the pharmaceutical composition comprises one or more pharmaceutically acceptable excipients.

5. The method of claim 1, wherein the first pharmaceutical composition and the second pharmaceutical composition are administered concurrently.

6. The method of claim 1, wherein the first pharmaceutical composition and the second pharmaceutical composition are administered sequentially.

7. The method of claim 1, wherein one or both of the first pharmaceutical composition and the second pharmaceutical composition are administered as a gas or aerosol.

8. The method of claim 1, wherein one or both of the first pharmaceutical composition and the second pharmaceutical composition are administered as solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, or elixirs.

9. A method of treating cancer in a subject, comprising the steps of:
administering to the subject a therapeutically effective amount of SP-2577; and
administering to the subject a therapeutically effective amount of a first pharmaceutical composition selected from the group consisting of Ruxolitinib, Dasatinib, Nilotinib, Imatinib, Bosutinib, and a drug targeting ABL2 pathway.

10. The method of claim 9, wherein the subject is human.

11. The method of claim 9, wherein the first pharmaceutical composition comprises one or more pharmaceutically acceptable excipients.

12. The method of claim 9, wherein the SP-2577 and the first pharmaceutical composition are administered concurrently.

13. The method of claim 9, wherein the SP-2577 and the first pharmaceutical composition are administered sequentially.

14. The method of claim 9, wherein one or both of the first pharmaceutical composition and the second pharmaceutical composition are administered as a gas or aerosol.

15. The method of claim 9, wherein one or both of the first pharmaceutical composition and the second pharmaceutical composition are administered as solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, or elixirs.

16. A pharmaceutical composition for the treatment of Ewing's Sarcoma, the pharmaceutical composition comprising:
a first active ingredient, wherein the first active ingredient is SP-2577; and
a second active ingredient, wherein the second active ingredient is selected from the group consisting of Ruxolitinib, Dasatinib, Nilotinib, Imatinib, Bosutinib, and a drug targeting ABL2 pathway.

17. The pharmaceutical composition of claim 16, further comprising at least one pharmaceutically acceptable carrier.

18. The pharmaceutical composition of claim 16, wherein the drug targeting ABL2 pathway is an ABL inhibitor.

19. The pharmaceutical composition of claim 16 formulated as a liquid, emulsion, or suspension.

20. The pharmaceutical composition of claim 16, formulated as a tablet, pill, pellet, capsule, capsule including liquid, or powder.
